(19) United States
(12) Patent Application Publication
(10) Pub. No.: US 2016/0030252 A1
(43) Pub. Date: Feb. 4, 2016

(54) Slip-Cut Operation with Static Electric Holding Force and Ultrasonic Bonding Apparatus

(71) Applicant: Curt G. Joa, Inc., Sheboygan Falls, WI (US)

(72) Inventors: Song Sayaovong, Milwaukee, WI (US); Donald R. Dodelin, Woodstock, GA (US); Jon Allen Pelland, Sheboygan, WI (US); Robert E. Andrews, Sheboygan, WI (US); Gottfried Jason Hohm, Sheboygan Falls, WI (US)

(21) Appl. No.: 14/815,567

(22) Filed: Jul. 31, 2015

Related U.S. Application Data
(60) Provisional application No. 62/032,727, filed on Aug. 4, 2014.

(51) Int. Cl. A61F 13/15

(52) U.S. Cl. CPC A61F 13/15804; A61F 13/15585; A61F 13/15723; A61F 13/15739; A61F 2013/15821; A61F 2013/15926

(57) ABSTRACT

A slip-cut mechanism uses static force to hold a material layer onto a preferably smooth rotating drum, and ultrasonic energy bonds discrete pieces of the material layer to second material layer.

### RELATED APPLICATIONS

This application claims the benefit of co-pending U.S. Provisional Patent Application Ser. No. 62/032,727 filed 4 Aug. 2014.

### BACKGROUND OF THE INVENTION

The present invention relates to disposable hygiene products and more specifically, to methods and apparatuses for processing disposable hygiene products. More specifically, the invention relates to cutting and applying segments of one web to attach to a disposable diaper.

This invention relates to a method and apparatus for rapidly and accurately transporting a discrete article or a web of material. This invention is not limited to its preferred use, carrying components of a disposable diaper or sheet of paper; but instead the methods and apparatus' of the present invention may be used in wide ranging applications.

Generally, diapers comprise an absorbent insert or patch and a chassis, which, when the diaper is worn, supports the insert proximate a wearer's body. Additionally, diapers may include other various patches, such as tape tab patches, reusable fasteners and the like. The raw materials used in forming a representative insert are typically cellulose pulp, tissue paper, poly, nonwoven web, acquisition, and elastic, although application specific materials are sometimes utilized. Usually, most of the insert raw materials are provided in roll form, and unwound and applied in continuously fed fashion.

In the creation of a diaper, multiple roll-fed web processes are typically utilized. To create an absorbent insert, the cellulose pulp is unwound from the provided raw material roll and de-bonded by a pulp mill. Discrete pulp cores are created using a vacuum forming assembly and placed on a continuous tissue web. Optionally, super-absorbent powder may be added to the pulp core. The tissue web is wrapped around the pulp core. The wrapped core is debulked by proceeding through a calender unit, which at least partially compresses the core, thereby increasing its density and structural integrity. After debulking, the tissue-wrapped core is passed through a segregation or knife unit, where individual wrapped cores are cut. The cut cores are conveyed, at the proper pitch, or spacing, to a boundary compression unit.

While the insert cores are being formed, other insert components are being prepared to be presented to the boundary compression unit. For instance, the poly sheet is prepared to receive a cut core. Like the cellulose pulp, poly sheet material is usually provided in roll form. The poly sheet is fed through a splicer and accumulator, coated with an adhesive in a predetermined pattern, and then presented to the boundary compression unit. In addition to the poly sheet, which may form the bottom of the insert, a two-ply top sheet may also be formed in parallel to the core formation. Representative plies are an acquisition layer web material and a nonwoven web material, both of which are fed from material parent rolls, through a splicer and accumulator. The plies are coated with adhesive, adhered together, cut to size, and presented to the boundary compression unit. Therefore, at the boundary compression unit, three components are provided for assembly: the poly bottom sheet, the core, and the two-ply top sheet.

A representative boundary compression unit includes a profiled die roller and a smooth platen roller. When all three insert components are provided to the boundary compression unit, the nip of the rollers properly compresses the boundary of the insert. Thus, provided at the output of the boundary compression unit is a string of interconnected diaper inserts. The diaper inserts are then separated by an insert knife assembly and properly oriented, such as disclosed in co-pending U.S. Application No. 61/426,891, owned by the assignee of the present invention and incorporated herein by reference. At this point, the completed insert is ready for placement on a diaper chassis.

A representative diaper chassis comprises nonwoven web material and support structure. The diaper support structure is generally elastic and may include leg elastic, waistband elastic and belly band elastic. The support structure is usually sandwiched between layers of the nonwoven web material, which is fed from material rolls, through splicers and accumulators. The chassis may also be provided with several patches, besides the absorbent insert. Representative patches include adhesive tape tabs and resealable closures.

The process utilizes two main carrier webs; a nonwoven web which forms an inner liner web, and an outer web that forms an outwardly facing layer in the finished diaper. In a representative chassis process, the nonwoven web is slit at a slitter station by rotary knives along three lines, thereby forming four webs. One of the lines is on approximately the centerline of the web and the other two lines are parallel to and spaced a short distance from the centerline. The effect of such slitting is twofold; first, to separate the nonwoven web into two inner diaper liners. One liner will become the inside of the front of the diaper, and the second liner will become the inside of the back of that garment. Second, two separate, relatively narrow strips are formed that may be subsequently used to cover and entrap portions of the leg-hole elastics. The strips can be separated physically by an angularly disposed spreader roll and aligned laterally with their downstream target positions on the inner edges of the formed liners. This is also done with turn bars upon entrance to the process.

After the nonwoven web is slit, an adhesive is applied to the liners in a predetermined pattern in preparation to receive leg-hole elastic. The leg-hole elastic is applied to the liners and then covered with the narrow strips previously separated from the nonwoven web. Adhesive is applied to the outer web, which is then combined with the assembled inner webs having elastic thereon, thereby forming the diaper chassis. Next, after the elastic members have been sandwiched between the inner and outer webs, an adhesive is applied to the chassis. The chassis is now ready to receive an insert.

In diapers it is preferable to contain elastics around the leg region in a cuff to contain exudates for securely within the diaper. Typically, strands of elastic are held by a non-woven layer that is folded over itself and contains the elastics within the overlap of the non-woven material. The non-woven is typically folded by use of a plow system which captures the elastics within a pocket, which is then sealed to ensure that the elastics remain in the cuff.

Most products require some longitudinal folding. It can be combined with elastic strands to make a cuff. It can be used to overwrap a stiff edge to soften the feel of the product. It can also be used to convert the final product into a smaller form to improve the packaging.

To assemble the final diaper product, the insert must be combined with the chassis. The placement of the insert onto the chassis occurs on a placement drum or at a patch applicator. The inserts are provided to the chassis on the placement drum at a desired pitch or spacing. The generally flat chassis/insert combination is then folded so that the inner webs face each other, and the combination is trimmed. A sealer bonds the webs at appropriate locations prior to individual diapers being cut from the folded and sealed webs.

Roll-fed web processes typically use splicers and accumulators to assist in providing continuous webs during web processing operations. A first web is fed from a supply wheel (the expiring roll) into the manufacturing process. As the material from the expiring roll is depleted, it is necessary to splice the leading edge of a second web from a standby roll to the first web on the expiring roll in a manner that will not cause interruption of the web supply to a web consuming or utilizing device.

In a splicing system, a web accumulation dancer system may be employed, in which an accumulator collects a substantial length of the first web. By using an accumulator, the material being fed into the process can continue, yet the trailing end of the material can be stopped or slowed for a short time interval so that it can be spliced to leading edge of the new supply roll. The leading portion of the expiring roll remains supplied continuously to the web-utilizing device. The accumulator continues to feed the web utilization process while the expiring roll is stopped and the new web on a standby roll can be spliced to the end of the expiring roll.

In this manner, the device has a constant web supply being paid out from the accumulator, while the stopped web material in the accumulator can be spliced to the standby roll.

Some diaper forming techniques are disclosed in co-pending U.S. application Ser. No. 12/925,033 which is incorporated herein by reference. As described therein, a process wherein a rotary knife or die, with one or more cutting edges, turns against and in coordination with a corresponding cylinder to create preferably trapezoidal ears. Ear material is slit into two lanes, one for a left side of a diaper and the other for a right side of a diaper. Fastening tapes are applied to both the right and the left ear webs. The ear material is then die cut with a nested pattern on a synchronized vacuum anvil.

The resulting discrete ear pieces however, due to the trapezoidal pattern of the ears, alternate between a correct orientation and an incorrect (reversed) orientation. The reversed ear is required to be rotated 180° into the correct orientation such that the ears and associated tape present a left ear and a right ear on the diaper.

To accomplish the reversal of the ear pattern, discrete ear pieces are picked up at the nested ear pitch by an ear turner assembly that will expand to a pitch large enough for ears to be unnested and allow clearance for every other ear to be rotated. The rotated ears are then unnested and into the correct orientation.

Two ear turner assemblies can be provided, to rotate every other ear applied to the right side of the product, and every other ear applied to the left side of the product. In this manner, for a single product, one of the two ears will have been rotated 180°.

Continual improvements and competitive pressures have incrementally increased the operational speeds of disposable diaper converters. As speeds increased, the mechanical integrity and operational capabilities of the applicators had to be improved accordingly.

Generally, diapers comprise an absorbent insert or patch and a chassis, which, when the diaper is worn, supports the insert proximate a wearer's body. Additionally, diapers may include other various patches, such as tape tab patches, reusable fasteners and the like. The raw materials used in forming a representative insert are typically cellulose pulp, tissue paper, poly, nonwoven web, acquisition, and elastic, although application specific materials are sometimes utilized. Usually, most of the insert raw materials are provided in roll form, and unwound and applied in assembly line fashion.

When manufacturing hygiene products, such as baby diapers, adult diapers, disposable undergarments, incontinence devices, sanitary napkins and the like, a common method of applying discrete pieces of one web to another is by use of a slip-and-cut applicator. A slip-and-cut applicator is typically comprised of a cylindrical rotating vacuum anvil, a rotating knife roll, and a transfer device. In typical applications, an incoming web is fed at a relatively low speed along the vacuum face of the rotating anvil, which is moving at a relatively higher surface speed and upon which the incoming web is allowed to “slip”. A knife-edge, mounted on the rotating knife roll, cuts off a segment of the incoming web against the anvil face. This knife-edge is preferably moving at a surface velocity similar to that of the anvil's surface. Once cut, the web segment is held by vacuum drawn through holes on the anvil's face as it is carried at the anvil's speed downstream to the transfer point where the web segment is transferred to the traveling web. In usual embodiments of slip-cut apparatus, an impact severing the film is accomplished with a knife-anvil arrangement.

Current slip-cut machines use two zones of vacuum on an anvil roll. The leasing edge of a patch is fed onto the “low vacuum” zone on the roll with it spinning at a surface velocity determined by the full product length—a higher speed than the incoming patch. The material slips on the vacuum until in passes into a high vacuum zone on the anvil roll. Simultaneously with the approach to the high vacuum roll the web is cut to form the trailing edge of the patch and that patch is not free to move at the full speed. It is usually combined with a full length web at this point (e.g. the nonwoven backsheet).

In diaper manufacturing equipment, there are processes in which nonwoven polymer webs and other materials such as pulp, adhesives, stretch films, etc., are formed, combined, cut, or transferred. These processes often require vacuum systems to hold the webs and other materials to transport conveyors, rotating drums and other materials being processed. In addition, adhesives, ultrasonic bonding systems, and mechanical bonding systems are used to hold materials together during processing. Although these processes are used today, they have high capital expense, high noise energy creation, high energy consumption, high consumable material expense, and possibly machine contamination.

One sought improvement is to minimize reliance on vacuum conveyors to transport either continuous webs of material or discrete components of disposable products. The current process uses high vacuum levels and a lot of air flow to hold the substrate to the belt or transporting pucks. This process holds and transfers sufficiently, but has high cost to acquire this vacuum and high cost for noise abatement.

### SUMMARY OF THE INVENTION

According to the present invention there is reduced or no vacuum and the leading edge of a web is accelerated on a relatively smooth roll by a static electric attraction force. When the trailing edge of the patch material is cut, the static force is sufficient to take that web away at the high speed consistent with the full product length.

In one embodiment, a nonwoven with adhesive applied to it is attached to a patch of intermittent poly film.

The present invention allows for the use of static pinning in addition to or in place of vacuum systems to hold materials against conveyors, rotating drums, and other transport equipment. Static technology could also be used to bind fiberized pulp, super absorbent polymer, and fine strings of adhesive. These actions would prevent these materials from becoming airborne and contaminating the machine and the products being manufactured by the machinery.

In an alternate embodiment, an ultrasonic bonding step can take place away from the static pinning roll, such as downstream of a cutting unit. This allows for independent tuning of each unit. Additionally, the ultrasonic bonding may become independent of product size and reduce costs on machines that run multiple sizes. Furthermore, it allows for a common ultrasonics package across multiple platforms and sizes. Alternatively, if a bonding step is desired at a static pinning anvil, it could be adhesive, ultrasonic, or a combination of both; or it could be a temporary bond such as a tackdown to control layers until reaching the final bonding station further downstream.

In another embodiment, minimal or no adhesive and an ultrasonic bond would be applied to each edge of a patch using a one or a pair of rotary horns. This would tack down both edges and then the leading edge of the patch would be tacked down against the nonwoven using a stationary or a rotary ultrasonic horn. If desired, the stationary or rotary horn could bond more than just the leading edge of the patch; perhaps even the entire area, or shaped areas.

According to the present invention, the gap between ultrasonic horns can be varied in the ultrasonic lamination so there is diminished risk of pinholes (if they are not desired) created by ultrasonic bonding, and the unbounded areas of a laminate can stretch over a broader area and resist puncture by sharp superabsorbent (SAP) particles.

In another aspect of the present invention, a move away from usual severing techniques of a knife-anvil arrangement is accomplished by using a hot wire cutting technique or other nonimpact severing techniques such as water jet, laser, electro discharge cutting or perforation, which has the added benefit of suppressing vibration.

In this ultrasonic embodiment, it is preferable to select poly backsheet materials which are thermally compatible with a companion substrate. For instance, if a conventional spunbond polypropylene or SMS is used, the poly would have a majority of polypropylene in the layer contacting the nonwoven, if a polyethylene/polypropylene bicomponent nonwoven is used, the film can have a majority of polyethylene resin(s) in the layer contacting the nonwoven.

The present invention has the ability to smoothly lay down a cut piece without foldovers of the corners. In the usual vacuum mode there can be areas without vacuum holes and in those areas there is nothing to prevent the film from turning upward leaving corner flaps, which can be undesirable. Additionally, the ultrasonic embodiment reduces an ongoing adhesive material cost.

### BRIEF DESCRIPTION OF THE DRAWINGS

FIGS. 1-6 generally represent a prior art apparatus and method of conveying and handing off articles by providing high speed vacuum porting to selected vacuum pattern areas on a rotating cylindrical roll.

FIG. 1 is a diagrammatic side view of a Prior Art process.

FIG. 2 is a top view of a prior art ear forming web including an individual ear detached from the web.

FIG. 3 is a front view of a prior art anvil roll.

FIG. 4 is a perspective view of a prior art anvil roll.

FIG. 5 is a cross sectional view of the prior art anvil roll.

FIG. 6 is a side view of the prior art anvil roll, showing an endface of the anvil, and a vacuum manifold pattern applied to vacuum holes disposed on the endface of the anvil.

FIG. 7 is a side schematic view of a slip/cut knife and anvil mechanism which uses static energy applied to a material layer to hold a material layer and discrete pieces of the material layer onto a preferably smooth rotating drum, and ultrasonic energy bonds discrete pieces of the material layer to second material layer.

FIG. 8 is a top view of a bonding pattern of discrete pieces of the material layer bonded to the second material layer.

FIG. 9a is a top view of an alternate bonding pattern of discrete pieces of the material layer bonded to the second material layer;

FIG. 9b is a top view of a second alternate bonding pattern of discrete pieces of the material layer bonded to the second material layer;

FIG. 10 is a side schematic view of a slip/cut hot knife and anvil mechanism which uses static energy applied to a material layer to hold a material layer and discrete pieces of the material layer onto a preferably smooth rotating drum, and ultrasonic energy bonds discrete pieces of the material layer to second material layer.

FIG. 11 is a side schematic view of a slip/cut hot knife and anvil mechanism with troughs provided on an otherwise preferably smooth rotating drum.

FIG. 12 is a side schematic view of a slip/cut static cutting and anvil mechanism.

FIG. 13 is a side schematic view of an eccentric overrunning hot knife severing mechanism.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention.

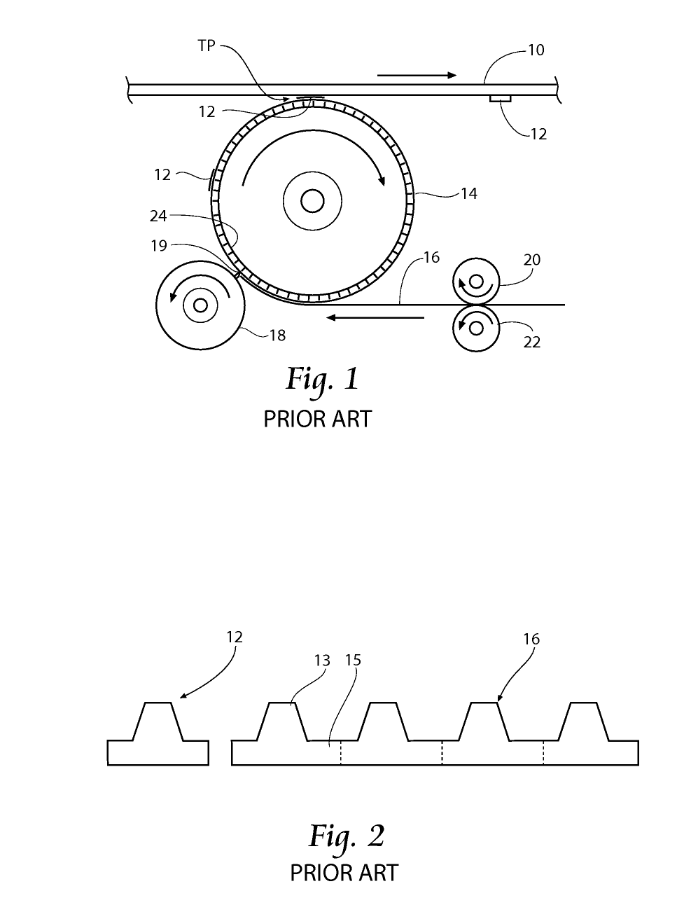

FIGS. 1-6 generally describe a rotating anvil using vacuum to hold discrete pieces on a moving web, in the prior art. High speed vacuum is also used in a similar manner to transport discrete pieces and entire webs on flat conveyors through the manufacturing process. The present invention relates to transporting discrete pieces and entire webs through both rotation and flat conveyance by minimizing the vacuum usage shown in FIGS. 1-6 and on prior art flat vacuum conveyors (not shown).

Referring to the drawings there is seen in FIG. 1 a diagrammatic illustration of a prior art process for applying tabs to webs in a diaper making process. This prior art method of affixing the segments 12 to the web 10, with a different anvil, the new anvil 114 described below. Web 10 is a composite material used in formation of diapers which is generally formed of various layers of material such as plastic back sheets, absorbent pads and nonwoven topsheets. A series of ears 12 are applied to web 10. In the illustrated process a rotatable vacuum anvil 14 is used to supply the ears 12 to web 10. Anvil 14 has internally reduced air pressure or vacuum (not shown), and a plurality of openings 24 are provided through its surface to enable suction of the tab segments 12 against the anvil surface 14. A web of the ear tab forming material 16 is fed by rollers 20 and 22 against the anvil surface 14 where it is cut into segments by a rotary knife 18.

In the prior art, the surface of the anvil roll 14 has vacuum holes 24 on its smooth surface. In a typical configuration of a slip-and-cut applicator, there is a pattern of vacuum holes 24 distributed to evenly draw the entering web onto the surface of anvil 14 and thence into the cut point where the knife edge 18 engages the anvil 14.

It can be seen from FIG. 1 that in the prior art, the infeed of the ear tab forming material 16 can be at a first speed (with individual ears 12 spaced together), after which the individual ears gain speed to the speed of the anvil 14. Typical infeed speeds could be 120 mm/product for the infeed, while anvil speeds could be 450 mm/product on the anvil. This transition from the slower first speed to the quicker second speed takes place at the cut point, the ear tab forming material 16 slipping on the anvil 14 until cut. However, immediately at the transition cut point 18 from the slower speed to the faster speed, it is desired to place vacuum on the ears because centrifugal force would try to throw the ears off of the vacuum anvil 14.

A continuous ear forming web 16 is provided to the system. The web 16 is comprised of two portions, 12a and 12b, as shown in FIG. 2. Segment 12a is more specifically referred to as the tab section of the ear 12, segment 12b is the ribbon section of the ear 12. The ear forming material 16 is cut into individual ears 12 by the rotary knife 18 as shown in FIG. 1, along lines such as the dashed lines shown in FIG. 2.

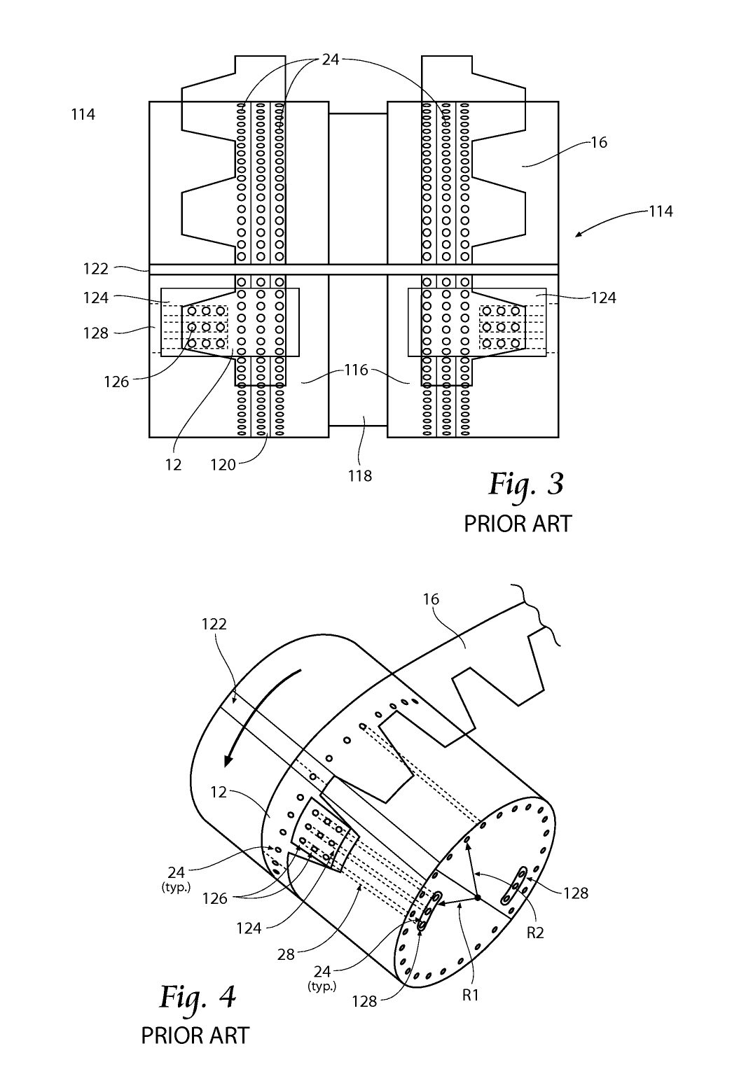

Referring now to FIG. 3, a front view of an anvil roll 114 of the prior art is shown carrying ear forming material 16 (and later, an ear 12) in phantom. The anvil roll 114 is preferably formed with two vacuum portions 116 separated by a center groove portion 118. The vacuum portions 116 are preferably mirror images of each other. The anvil roll 114 is symmetrical about a center plane through its circumference. Each vacuum portion 116 contains several circumferential rows of circular vacuum holes 24. Each vacuum portion 116 may also contain a circumferential groove 120 with an additional circumferential row of vacuum holes 24 located in the circumferential groove 120.

The preferred embodiment of the anvil roll 114 of the prior art is also formed with two diametrically opposed anvil pockets 122 and two diametrically opposed pairs of ear retaining portions 124. The ear retaining portions can be created as inserts, with different vacuum patterns applied as the user deems necessary. Each anvil pocket 122 is a groove which extends across the face of the entire anvil roll 114. One ear retaining portion 124 is located on each of the vacuum portions 116. Each ear retaining portion 124 has an ear vacuum hole pattern 126 made of a plurality of vacuum holes 24 located at or near the surface of the anvil roll 144. The preferred embodiment, as shown in FIG. 3 is a plurality of rows of vacuum holes 24, each row having a plurality of vacuum holes 24, although more or less than those configurations or patterns shown can be used.

In operation, two webs of ear material 16 are carried by the anvil 114. One web of ear material 16 is located on each vacuum portion 116. A single ear 12 is cut from the ear web 16 when the rotary knife 18 engages the anvil roll 114 at the anvil pocket 122. Immediately after a single ear 12 is cut from the ear web 16, the single ear 12 is located on the ear retaining portion 124, particularly the tab portion 12a of the ear 12 as shown in FIG. 2. At this point the vacuum in the ear retaining portion 124 has been engaged to secure the single ear 12 to the anvil roll 114. As the anvil roll 114 rotates the vacuum is released at a predetermined location so that the single ear 12 can be applied to the diaper web 10. Because this configuration has two vacuum portions 116, a pair of two ears 12 is cut each time the rotary knife 18 engages the anvil roll 114. This allows for two pair of ears 12 to be cut with each revolution of the anvil roll 114. Shown in dotted line in FIG. 3 is a vacuum slot 128, described below.

Referring now to FIG. 4, a perspective view of the anvil 114 is shown. The anvil 114 will be described in relation to its endface and its outer surface, the outer surface that surface shown on FIG. 3 and the endface the two ends of the anvil 114.

The vacuum slot 128 contains a plurality of vacuum holes 24 that allow commutation of the vacuum to the entire ear vacuum hole pattern 126, allowing the pattern 126 to be activated simultaneously, as opposed to each of the rows that comprise the vacuum of vacuum holes 24 being enabled one at a time. The vacuum pattern 126 is activated utilizing drilled ports 28 that communicate the vacuum from the slot 128 to the individual holes 24 of the pattern 126. It should be noted that the pattern 126 can also be provided with a depressed slot configuration so that it too is all simultaneously enabled with vacuum.

The remaining vacuum holes 24 provided on the anvil roll 114 are enabled sequentially, by known vacuum commutation method utilizing cross drilled ports 28.

The vacuum slot 128 is provided at a first radius R1 on the anvil roll 114, the remaining vacuum holes provided at a different R2. The differing radii R1 and R2 allow two vacuum manifolds (not shown) to communicate each at a different radius, R1 or R2, thus selectively applying vacuum to the anvil.

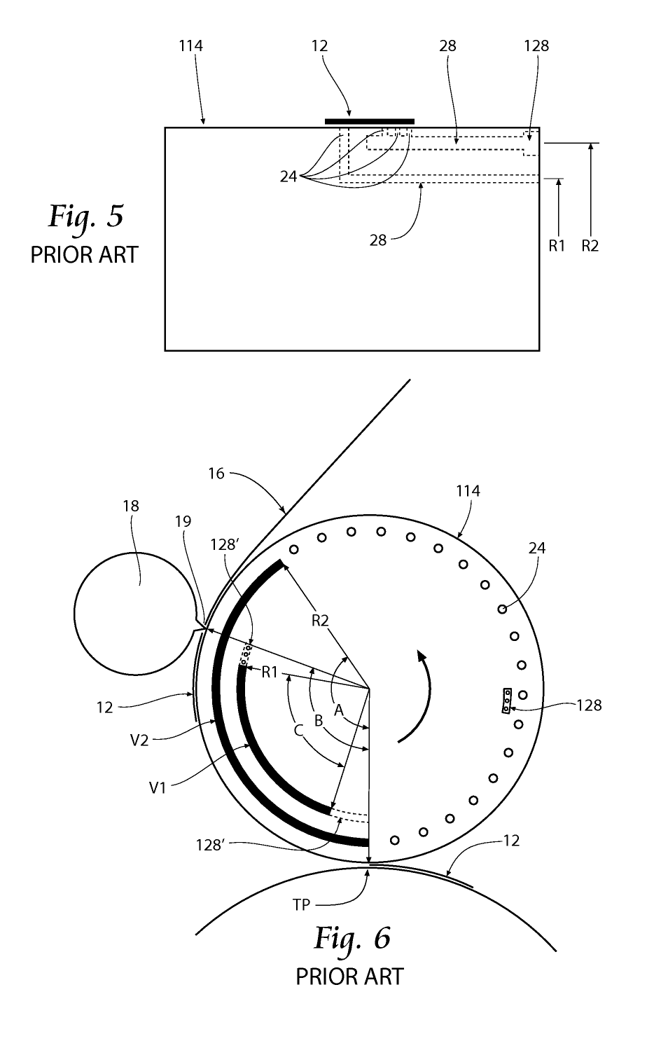

Referring now to FIG. 5, a cross sectional view of the anvil roll 114 of the prior art is shown. In this embodiment, the slot 128 has been placed at R2. It is appreciated that the slot 128 communicating with the pattern 126 can be placed at either R1 or R2, and the remaining vacuum holes 24 communicating with drilled ports 28 can be interchanged at either R1 or R2. For machining purposes, it is likely preferable to place the slot 128 communicating with the pattern at R2 for simplicity in machining.

Referring now to FIG. 6, a side view of the anvil roll 114 is shown, showing the endface of the anvil, or the circular portion of the cylindrical body 114. The ear web 16 is shown infeeding to the anvil 114, where it is then cut with the rotary knife 18. It is desired to apply the vacuum to the pattern 126 simultaneously with the knife cut.

The range of vacuum application is provided for with a manifold (not shown) that continuously applies vacuum to vacuum patterns V1 and V2. Vacuum pattern V1 is at R1, Vacuum pattern V2 is at R2. Vacuum pattern V1 applies vacuum to the slot 128 each time the slot 128 rotates through the vacuum pattern V1 provided on the manifold. When the slot 128 is in communication with V1, vacuum is applied to vacuum holes 24 associated in the slot 128 on the endface of the anvil for commutation to the pattern 126 on the outer surface of the anvil 114. When the slot 128 is not in communication with V1, the vacuum to the pattern 126 is turned off.

Vacuum pattern V2 is applied to the vacuum holes 24 disposed on the endface of the anvil 114 and the associated circumferential ribbon vacuum hole pattern on the outer surface of the anvil 114 throughout V2. As each successive vacuum hole 24 rotates through V2, the vacuum is on. As each successive vacuum hole 24 leaves V2, its vacuum is turned off.

From the center of the endface, a radius extending to the contact point of the knife 18 with the anvil roll 114 can be extended, and as the anvil roll rotates through angle B as shown, the rotation of the ear 12 will be from the knife point to the transfer point TP. It is throughout this angle B that vacuum is desired across the pattern 126 and onto the ear 12. To accomplish this, a smaller angle C has vacuum applied to it. The angle C can be expressed mathematically as the angle B minus twice the width 128′ of the slot 128. This is because pattern 126 is placed in communication with the slot 128, the slot 128 communicates vacuum simultaneously to the pattern 126. Therefore, the leading edge of the ear 12 and the trailing edge of the ear 12 will receive vacuum at the same time. Therefore, the user must allow the leading edge of the ear 12 to pass by the knife 18 the desired length of the ear 12 prior to engaging the vacuum onto the ear 12. Similarly, prior to arriving at the transfer point TP, the vacuum will have to be released on both the leading and trailing edges of the ear 12 simultaneously, allowing the ear 12 to continue on its downstream path.

An angle A, larger than angle B, is provided to define V2, as it is desired to draw the web 16 into contact with the anvil both prior to and during cutting by the knife 18.

Many of the same transport functions are accomplished by the present invention using conveyance techniques accomplished without vacuum or with minimal assisted vacuum. Both flat and rotational conveyance are contemplated in the description below.

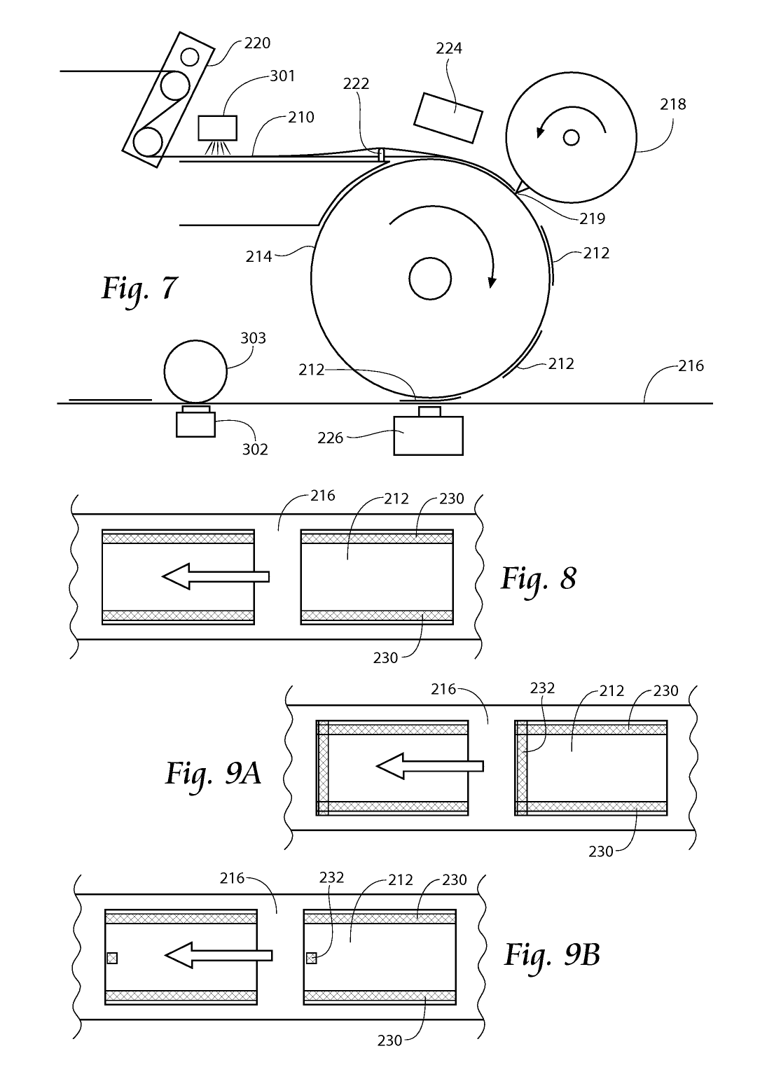

Referring now to FIG. 7, a slip-cut unit with a static electric holding force and ultrasonic bonding apparatus is shown. The units shown in FIG. 7 and following figures are particularly well suited, but not limited to use for processing poly web materials. Poly is a stretchy material, so traditional slip/cut techniques are difficult to employ because the poly material may stretch under tension of vacuum anvil prior to the trailing edge being severed. At the moment of cut, the leading edge of poly material is also difficult to control, and the poly material may snap back slightly during a traditional slip/cut technique.

One particular application of the units shown in FIG. 7 and following figures is use of a poly material to roughly match the size of an absorbent core insert. In some prior art designs, the entire chassis is provided with poly material. To save on material costs and reduce waste, it may be preferable to use poly only underlying the absorbent insert. Minimizing poly use also provides for more breathability.

Still referring to FIG. 7, a material layer 210 (for instance poly material) first passes through an optional rotary accumulator 220, which with servo and logic settings optimized, controls the infeed rate of material 210 to the rotating drum 214 and knife roll 218 carrying knife 219. Static charger 224 applies a static charge to web 210 proximal to the entrance of web 210 onto rotating drum 214. Rotating drum 214 is preferably smooth to best carry charged web 210. Alternatively, rotating drum 214 bears a modest texture as to not interfere with desired slipping of material layer 210 across the face of drum 214. If desired to minimize material wrinkle, a wrinkle reducing device 222 can be provided prior to application of web 210 onto rotating drum 214. As discrete web pieces 212 are severed from web 210, they match speed with rotating drum 214, which is faster than the infeed rate of web 210. Discrete web pieces 212 can then be coupled or bonded to incoming web 216, by ultrasonic bonding unit 226, utilizing either fixed, rotary, and/or textured horns, acting through web pieces 212 and incoming web 216 and upon rotating drum 214 to form machine direction bonds 230 (FIG. 8).

In one preferred embodiment, discrete pieces 212 are bonded roughly at cross-machine direction (CD) edges of the discrete pieces 212, with little or no bonding of discrete pieces 212 in the CD center of patch, as shown in FIG. 8. It may be preferable to provide a CD bond 232 as shown in FIG. 9a to reduce airflow through the underside of web pieces 212 during downstream processing, or to bond across one ultrasonic horn width as shown in FIG. 9b. In this case, an additional bonding step or unit could be used either as part of bonding unit 226, or provided separately, for instance by a fixed, rotary and/or textured ultrasonic horn, or by a bladed horn.

Referring again to FIG. 7, an adhesive applicator 301 can be used like shown. In an alternative to, or as an auxiliary to, locating ultrasonic bonding unit 226 beneath drum 214, an ultrasonic bonding horn 302 and ultrasonic bonding anvil 303 can be located downstream of the cutting unit 218/drum 214 combination. This positioning of the horn 302 and anvil 303 can allow for independent tuning of the ultrasonic combination 302/303, and independent tuning of the cutting unit/drum combination 218/214. Additionally, the ultrasonic combination 302/303 may become independent of product size and reduce costs on machines that run multiple sizes. Furthermore, ultrasonic combination 302/303 allows for a common ultrasonics package across multiple platforms and machine and product sizes.

Adhesive from applicator 301 is optionally applied in patterns as shown generally in FIGS. 8, 9a, and 9b.

Referring specifically to bonding unit 226, an ultrasonic bond 230 is applied to each edge of the patch 212 using a pair of rotary horns. Bonding unit 226 tacks down both edges of patch 212, and then the leading edge of the patch 212 can be tacked down against the nonwoven 216 using a stationary ultrasonic horn. If desired, a stationary horn at bonding unit 226 could bond more than just the leading edge of the patch; perhaps even the entire area of the patch 212. Using bonding unit 226 desirably reduces the use and expense associated with traditional adhesives.

In addition, the present method and apparatus allows to smoothly lay down a cut piece 212 without foldovers of the corners. In the usual vacuum mode there can be areas without vacuum holes and in those areas there is nothing to prevent the film from undesirably turning upward leaving corner flaps.

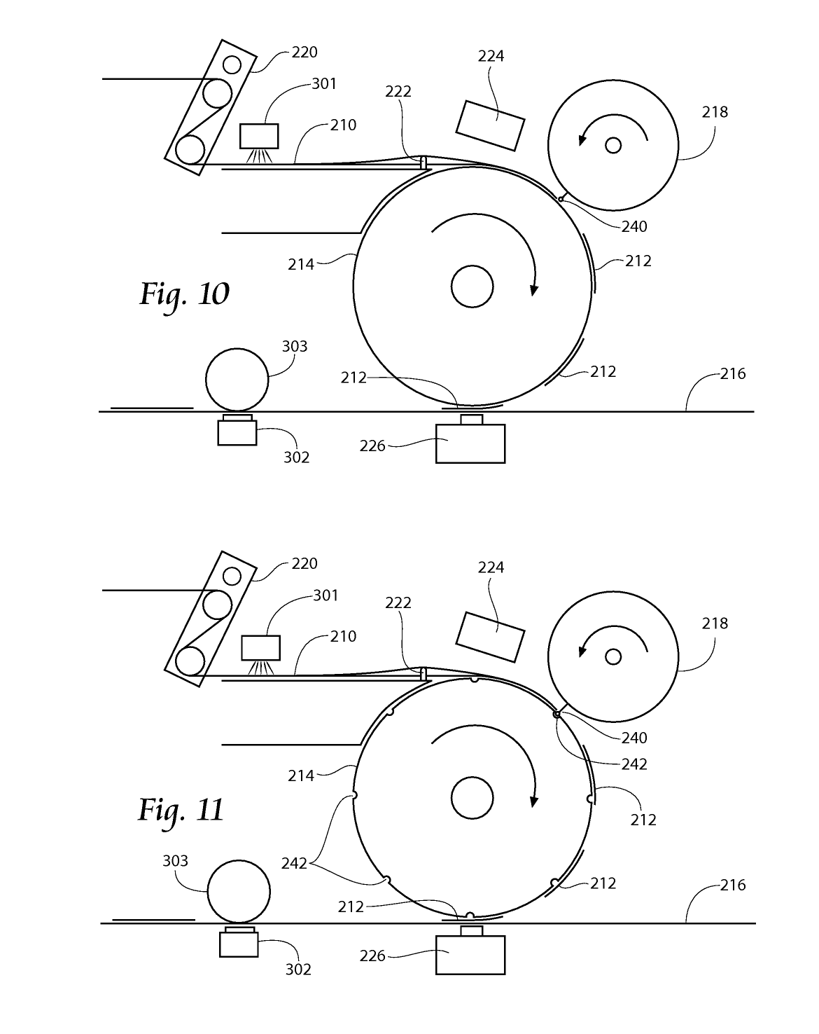

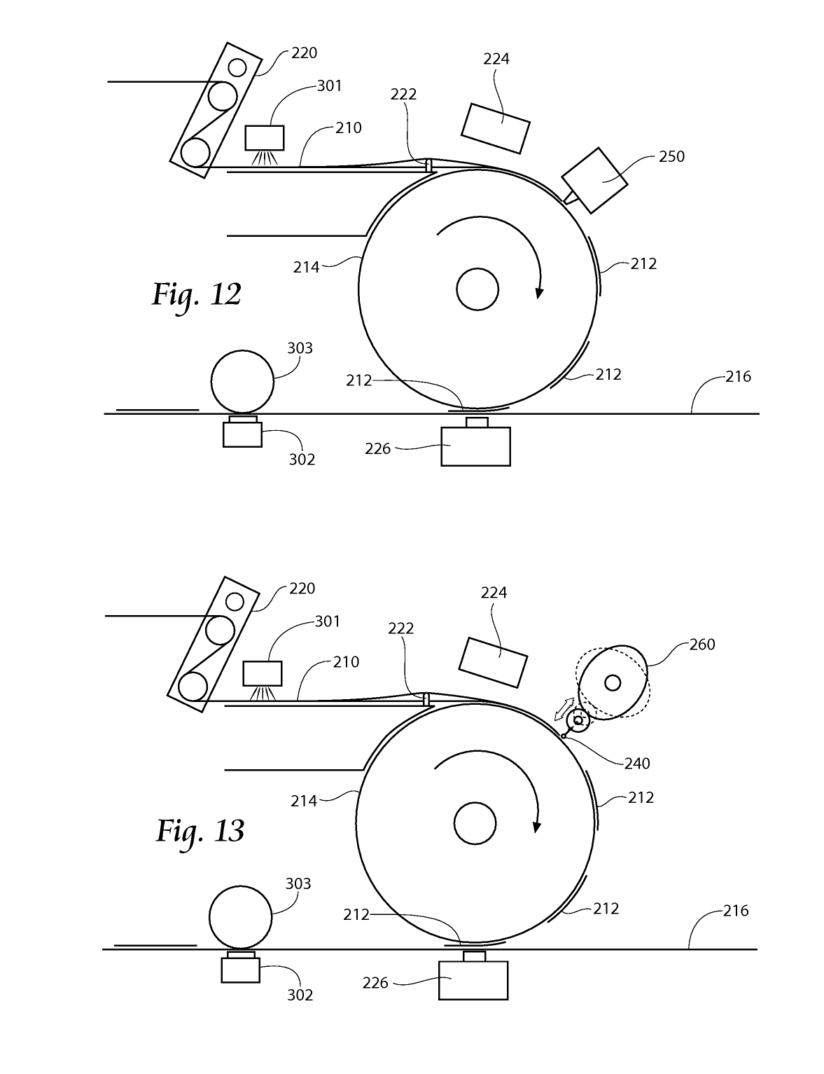

Referring now to FIG. 10, in an alternative embodiment, a hot knife 240 can be used in place of knife 219. Hot knife 240 can pass on or in close proximity to web 210 to create discrete pieces 212. In an alternate embodiment shown in FIG. 11, rotating drum 214 can be fitted with troughs 242 spaced apart on the surface of drum 214 to intermittently receive hot knife 240. In an alternate embodiment shown in FIG. 12, a static cutter 250 could also be employed in place of knife 219. In place of static cutter 250, other cutting techniques such as laser perforation, water jet and other nonimpact technologies could be used.

In FIG. 13, hot knife 240 could also be led to cutting proximity to web 210 by a combination orbital/suborbital unit 260 by eccentric overrunning techniques. One possible eccentric technique is a sun/planetary gear configuration (not shown).

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. An apparatus for processing a web of material, the apparatus comprising:
an infeeding web of material traveling at a first speed;
a source of static electricity imparting static electricity upon said infeeding web of material;
a rotating drum traveling at a second speed;
said infeeding web of material clinging to said rotating drum due to said imparted static electricity.

2. An apparatus according to claim 1, said apparatus further comprising a severing mechanism to sever said infeeding web of material into discrete pieces.

3. An apparatus according to claim 1, said apparatus comprising a slip and cut device.
